(19) United States
(12) Patent Application Publication
(10) Pub. No.: US 2006/0246505 A1
(43) Pub. Date: Nov. 2, 2006

(54) Modulation of the synthesis of insulin

(76) Inventor: Reinhard Walther, Neuenkirchen (DE)

Correspondence Address: NIXON & VANDERHYE, PC, 901 NORTH GLEBE ROAD, 11TH FLOOR, ARLINGTON, VA, 22203, US

(21) Appl. No.: 10/526,468

(22) PCT Filed: Sep. 2, 2003
(86) PCT No.: PCT/EP03/09757

§ 371 Date: Sep. 29, 2005

(51) Int. Cl. A61K 38/22; A61K 48/00; G01N 33/53

(52) U.S. Cl. 435/7.1; 514/12; 514/44

(57) ABSTRACT

The invention particularly relates to the use of substances, which modulate the activity of the proteins casein kinase II (CK II) and 14-3-3 epsilon and/or of the PcG protein EED or which influence the binding of the proteins casein kinase II (CK II) and 14-3-3 epsilon, of the PcG protein EED and/or of a fragment thereof with the protein pancreatic duodenal homeobox-1 (PDX-1) that plays a decisive roll in the glucose-induced biosynthesis of insulin, for influencing the synthesis of insulin or the provision of insulin.

The present invention particularly relates to the use of substances that modulate the activity of the proteins casein kinase II (CK II), 14-3-3 epsilon and/or of the PcG protein EED or that influence binding of the proteins casein kinase II (CK II), 14-3-3 epsilon, PcG protein EED and/or of a fragment of the same with pancreatic duodenal homeobox 1 (PDX-1) protein, which plays a decisive role in glucose-induced insulin biosynthesis, for influencing the synthesis of insulin or the provision of insulin, respectively.

Within mammalian organisms, after a meal, insulin stored in secretory granules is released from the beta cells of the islets of Langerhans within the endocrine pancreas, this process being triggered by glucose loading. At the same time resynthesis of insulin takes place (transcription and translation). It has been shown that PDX-1 is involved in this as a transcription factor (McKinnon and Docherty, Diabetologia (2001), 44: 1203-1214), and that the signalling pathways that trigger transcription can be inhibited by wortmannin and SB 203580. At the moment, however, it is not known how the processes that lead to secretion of insulin are coupled to inducing resynthesis.

The task is therefore to make substances (active agents) available that effectively influence the provision of insulin, and are therefore suitable for treating diseases characterised by a reduction in insulin synthesis, or that are accompanied by such a reduction, for example diabetes.

Within the framework of the instant invention, surprisingly three proteins have been identified that bind to the transcription factor PDX-1, which plays a decisive role in glucose-induced insulin biosynthesis (cf e.g. Lottmann et al., Journal of Molecular Medicine (2001) 79:321-328). Direct PDX-1 activators are not yet known in the prior art.

According to the present invention, after induction by glucose, proteins were identified in an experimental cell system developed by the inventor, which proteins in this phase i) are phosphorylated themselves and ii) physically interact with the transcription factor PDX-1. PDX-1 itself is also phosphorylated by glucose induction, whereby it is important that bacterially expressed PDX-1 can only bind to the DNA and act as activator after phosphorylation.

In this respect CK II in the insulin producing cell represents a glucose-induced PDX-1 kinase. The casein kinase II is a widespread serine/threonine kinase. The holoenzyme is a tetramer composed of two alpha or alpha' subunits (or either one of these subunits) and two beta subunits (Lotzeman et al., Biochemistry 36 (1990) 8436-47). This allows insulin provision to be modulated according to the present invention by changing the activity of this enzyme.

The 14-3-3 proteins are described as regulator proteins that can bind key elements of signal transduction pathways in the cell (such as for example the transcription factor FKHR) and thereby deactivate them. Only by phosphorylation of the 14-3-3 proteins the binding is removed. In its unphosphorylated state, protein 14-3-3 epsilon binds the transcription factor PDX-1 rendering it inactive. After glucose induction, 14-3-3 epsilon is phosphorylated and can release the bound and inactive transcription factor PDX1 that then as an activating factor initiates insulin synthesis.

The EED protein is one of the transcriptional repressors, whereby histone deacetylases appear to participate in the repression process. According to the present invention the EED is a large isoform of the protein, mentioned in Sewalt et al., Mol. Cell. Biol. 18 (1998) 3586-95 (cf. FIG. 4).

The work presented here has successfully allowed the three proteins mentioned (CK II, 14-3-3 epsilon and EED) to be identified as essential regulative elements in glucose-induced insulin biosynthesis. The correlation as described above with regard to the transcription factor PDX-1, with which the proteins interact physically, further enables the identification of new active agents that can effectively influence the provision of insulin and the development of a new and more effective generation of diabetes therapeutics with fewer side effects. Screening can be carried out with suitable assays, such as binding assays, by means of which the influence of the interaction (binding) of said proteins with PDX1 can be directly examined, or else with an assay where the functionalisation of PDX1 (phosphorylation, DNA binding, transcription activation) is analysed under the influence of active agents. Establishing such assays is well known to the skilled person.

The subject-matter of the present invention is therefore the use of one or several proteins according to SEQ ID NO:4 and/or SEQ ID NO:6 and SEQ ID NO:8 (CK II), 10 (14-3-3 epsilon) and 12 (EED) or fragments of said proteins, for performing binding assays using a protein according to SEQ ID NO:2 (PDX-1), wherein the fragments bind to PDX1, for the identification of substances that influence (promote, inhibit, modulate) binding between the protein or proteins or fragment(s) and PDX-1.

The invention relates, amongst other things, to a process for identifying substances that are suitable for influencing interaction of a protein according to SEQ ID NO:4 and/or SEQ ID NO:6 and SEQ ID NO:8 (CK II), 10 (14-3-3 epsilon), of the protein EED, or a fragment thereof, with the protein according to SEQ ID NO:2 (PDX-1), where
a) The protein according to SEQ ID NO:4 and/or SEQ ID NO:6 and SEQ ID NO:8 (CK II), 10 (14-3-3 epsilon), the protein EED or a fragment of said proteins is labelled,
b) The protein according to SEQ ID NO:2 (PDX1) is labelled,
c) The labelled proteins from step a) and step b) are brought into contact with each other and a measurement is performed to determine the signal/signals of the label(s),
wherein the labels are so selected that interaction of labelled proteins from step a) and b) can be detected and distinguished from the isolated unlabelled proteins via alteration of the detection signal/detection signals,
d) The mixture from step c) is brought into contact with a substance to be examined, and
e) Another measurement is performed to determine the signal/signals of the label(s),
wherein the substance to be examined is a substance that influences the interaction, if the signal(s) of the label(s) measured in step e) differ(s) from the signal(s) of the label(s) measured in step c).

Also included is a process for identifying substances that are suitable for influencing interaction of a protein according to SEQ ID NO:4 and/or SEQ ID NO:6 and SEQ ID NO:8 (CK II), 10 (14-3-3 epsilon), of the protein EED, or a fragment of said proteins, with the protein according to SEQ ID NO:2 (PDX-1), where either
a) The protein according to SEQ ID NO:4 and/or SEQ ID NO:6 and SEQ ID NO:8 (CK II), 10 (14-3-3 epsilon), the protein EED, or a fragment of said proteins, or
b) The protein according to SEQ ID NO:2 (PDX-1) is immobilised on a microtiter plate,
c) The other protein in question is labelled and brought into contact with the immobilised protein, wherein the presence of an interaction between the proteins mentioned in a) and b) is confirmed by detecting the labelling after performing corresponding washing steps,
d) The proteins are brought into contact with the substance to be examined,
   wherein the substance to be examined is a substance influencing the interaction if, after addition of the substance to be examined and performing corresponding washing steps on the microtiter plates, the labelling is no longer detectable.

The active agents identified with the aid of the screening processes performed, can be used in the treatment of (patho)physiological conditions, where a decreased production of insulin relative to the normal value is observed.

The invention therefore relates to the use of a substance that influences the interaction of one or several proteins according to SEQ ID NO:4 and/or SEQ ID NO:6 and SEQ ID NO:8 (CK II), 10 (14-3-3 epsilon) and EED or fragments of said proteins with the protein according to SEQ ID NO:2 (PDX-1), for the manufacture of a pharmaceutical composition for treating diseases that are characterised by decreased synthesis of insulin or that are accompanied by such decrease.

Also included is the use of a substance that
b) Modulates the activity of the protein according to SEQ ID NO:4 and/or SEQ ID NO:6 and SEQ ID NO:8 (CK II), 10 (14-3-3 epsilon), and/or the protein EED,
b) Binds to the protein according to SEQ ID NO:4 and/or SEQ ID NO:6 and SEQ ID NO:8 (CK II), 10 (14-3-3 epsilon), the protein EED or to a fragment of said proteins,
b) Phosphorylates the protein according to SEQ ID NO:2 (PDX-1), 4 and/or 6 and 8 (CK II), 10 (14-3-3 epsilon) or the protein EED, or
b) Increases the proportion of the protein according to SEQ ID NO:4 and/or SEQ ID NO:6 and SEQ ID NO:8 (CK II),
   for the manufacture of a pharmaceutical composition for the treatment of diseases that are characterised by decreased synthesis of insulin or that are accompanied by such decrease.

Here a disease that is characterised by decreased synthesis of insulin or that are accompanied by decreased synthesis of insulin is understood to mean various forms of diabetes, such as, for example, diabetes mellitus.

The invention further relates to a process for the manufacture of a pharmaceutical composition for the treatment of diseases that are characterised by decreased synthesis of insulin or that are accompanied by such decrease, wherein a screening procedure as mentioned above is performed, and the identified substance, which is identified as a substance influencing the interaction of a protein according to SEQ ID NO:4 and/or SEQ ID NO:6 and SEQ ID NO:8 (CK II), 10 (14-3-3 epsilon), EED or a fragment of said proteins with the protein according to SEQ ID NO:2 (PDX-1), is formulated into a pharmaceutical composition, using suitable excipients and/or carriers.

The subject-matter of the invention are therefore also pharmaceutical compositions that contain a substance obtainable to a screening procedure as mentioned above, as well as pharmaceutically compatible excipients and/or carriers.

According to a particular embodiment, one or several proteins according to SEQ ID NO:4 and/or SEQ ID NO:6 and SEQ ID NO:8 (CK II), 10 (14-3-3 epsilon) and EED and/or fragments of said proteins can be used for the manufacture of a pharmaceutical preparation for the treatment of a disease that is characterised by decreased synthesis of insulin or that is accompanied by such decrease.

The invention also relates to the use of one or several nucleic acids according to SEQ ID NO: 3 and/or 5 and 7 or 9, and/or one or several nucleic acids that encode EED, for the manufacture of a pharmaceutical preparation for the modulation of insulin synthesis in an individual. These preparations can find application, for example, in gene therapy, for instance in the generation of artificial, insulin producing cells for transplantation.

Within the framework of the work performed, the protein (EED), representing an EED isoform (cf. FIG. 4 in Sewalt et al. Mol Cell Biol (1998), 18(6): 3586-95), was identified for the first time as a regulative element which also plays an important role in insulin biosynthesis. According to the present invention, fragments of the proteins mentioned above are included, wherein the term EED also encompasses the shorter isoforms of EED (cf. FIG. 15).

Based on the findings, assays for measuring the functionalisation of PDX-1 can be constructed, giving information about the property of substances to inhibit or promote, as the case may be, binding of PDX-1 to the promotor of the insulin gene. In this way, using standard molecular biology procedures, transgenic cell cultures can for instance be established, where a reporter gene is introduced, whose gene product is easily detected and quantified, and which is expressed in a stable fashion under the control of a promotor with DNA sequences that bind PDX-1. Under inductive conditions—that is, the binding of PDX-1 to the promotor—the expression of the reporter gene can be analysed under influence of the substance to be tested.

The invention is now elucidated by means of examples.

### EXAMPLES

Material and Methods

Identification of Glucose-Induced Phosphylated Interaction Partners of PDX1:

MIN6 cells were cultivated to 80% confluence in DMEM, containing 25 mM glucose, 10% horse serum and 2.5% FCS (foetal calf serum). The cells were washed twice and were allowed to starve for three hours in Krebs Ringer Buffer (118 mM sodium chloride, 4.75 mM potassium chloride, 1.25 mM calcium chloride, 1.2 mM magnesium chloride, 0.05% (w/v) BSA, 25 mM sodium hydrogen carbonate, 10 mM Hepes, pH 7.4). After preincubation of the cells for three hours, these were equilibrated with 500 μCi/ml (³²P)-phosphoric acid (ICN) for an hour, and then incubated in the presence or absence of 16 mM glucose with and without the kinase inhibitors wortmannin (100 nM, 10 min preincubation) and SB203580 [(4-(4-fluorophenyl)-2-(4-methylsulphinylphenyl)-5-(4-pyridyl)-imidazole] (10 mM, 30 min preincubation) for a further 30 minutes. Next, the cells were washed with ice cold PBS and harvested. The cells were centrifuged for 30 seconds and resuspended in 100 μl 20 mM Hepes 7.8, 50 mM potassium chloride, 1% Triton X100, 0.1 mM EDTA, 20 mM β-glycerophosphate, 0.1 mM Na₃PO₄, complete mini-protease inhibitor cocktail (Roche). The cells were allowed to swell for 10 minutes on ice and were then centrifuged at 14,000 rpm. The supernatant (cytoplasmic extract) was transferred to a fresh Eppendorf tube, and the proteins were precipitated using acetone, then washed several times in acetone, dried and solubilised in lysis buffer (8 M urea, 2 M thiourea, 65 mM CHAPS, 120 mM DTT, 80 mM Tris). For the preparative gels, the labelled extracts were mixed with 4 mg of non-radioactive cytoplasmic extracts that had been treated in a similar fashion. Protein separation took place using an immobilised pH gradient of 3 to 10 (IPG strips, 18 cm, Amersham Biosciences) in an IPGphor isolectric focusing system (Amersham Biosciences). After equilibration of the strip in SDS buffer, the SDS-PAGE second dimension was performed over night using a 12.5% acrylamide gel. The preparative gels were stained using colloidal coomassie R-250 and subjected to autoradiography using a Phosphorus Imager (Molecular Dynamics). The analytic gels were subjected to autoradiography. So-called “pulldown experiments” were performed using a bacterially expressed GST-PDX1 protein as a bait in the cytoplasm of the glucose-stimulated ³²P-labelled MIN6 cells. After adding 5 μg GST for the saturation of unspecified binding sites, GST-PDX1 fusion protein, coupled to gluthatione agarose beads, was incubated with 100 mg of cytoplasmic extract with constant agitation for three hours at 4° C. After this incubation phase, the beads were pelleted at 3,000 g for 5 minutes, and the supernatant was collected and used for two-dimensional gel electrophoresis. To verify the interaction of phosphoproteins with the GST-PDX-1 protein, the pelleted beads were washed 3 times, suspended in lysis buffer, and then subjected to two-dimensional gel electrophoresis. Immunofluorescent staining and Western blotting were used with standard molecular biology protocols.

Results:

In the autoradiograph of the analytic gel from the “pulldown” (FIG. 5, A), spots of phosphorylated proteins interacting with PDX-1 were detected. It was possible to assign these spots to spots that had been coomassie-stained in preparative gels and they were identified by means of mass spectrometry (MALDI-TOF).

### DESCRIPTION OF THE FIGURES

FIG. 1:

Schematic representation of the identification of glucose-induced interaction partners of PDX-1

FIG. 2:

The effects of glucose on the subcellular localisation of endogenous PDX-1. A: The cells were incubated in Krebs Ringer Buffer for four hours with 0 mM glucose. B: The culture was then reincubated for 30 minutes with 16 mM glucose. Endogenous PDX-1 was detected using a polyclonal anti-PDX1 antiserum.

FIG. 3:

At high glucose concentrations, PDX-1 is modified in MIN6 cells. Western blot analysis of nuclear and cytoplasmic extracts that were produced from MIN6 cells, incubated in Krebs Ringer Buffer with 0 mM glucose (lane 1) and then transferred to 16 mM glucose (lane 2).

FIG. 4:

Silver staining of bacterially expressed GST-PDX-1. 5 μg (lane 1) and 1 μg (lane 2) purified GST-PDX-1; expressed in E. coli and separated using polyacrylamide gel electrophoresis.

FIG. 5:

Bacterially expressed GST-PDX-1 precipitates phosphoproteins from the cytoplasm of ³²P-labelled, glucose-stimulated MIN6 cells.
A: Mapping of phosphoproteins obtained using two-dimensional gel electrophoresis after a GST-PDX1 pulldown experiment.
B: Cytoplasmic phosphoproteins that were separated using two-dimensional gel electrophoresis after acetone precipitation.

FIG. 6:

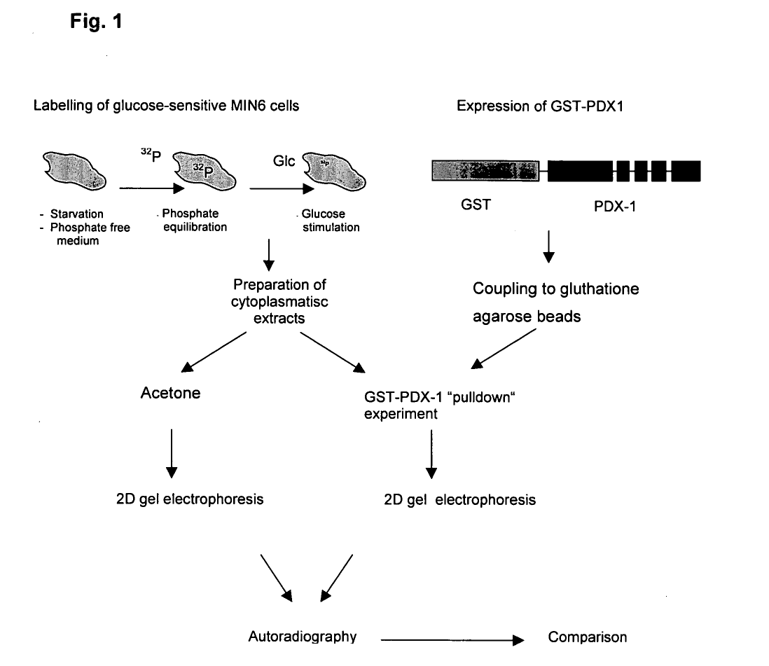

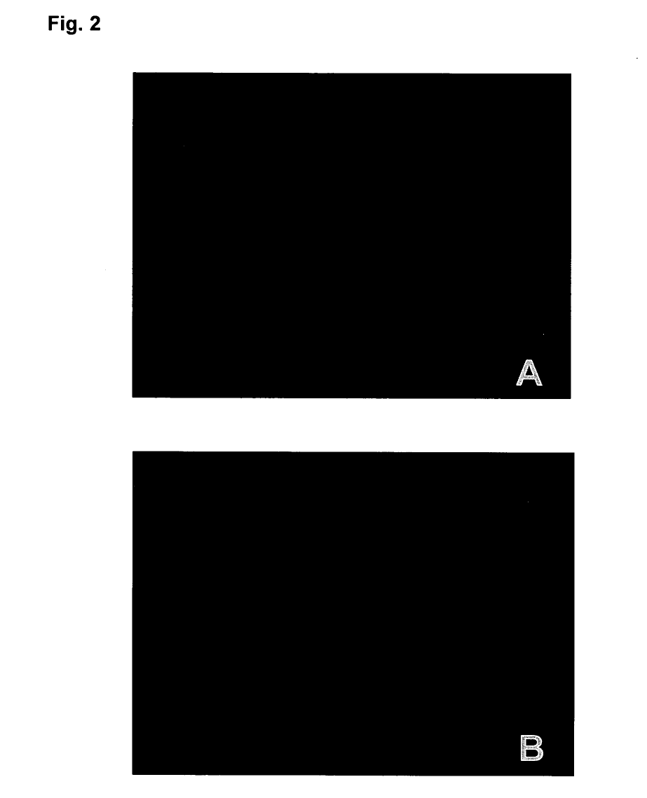

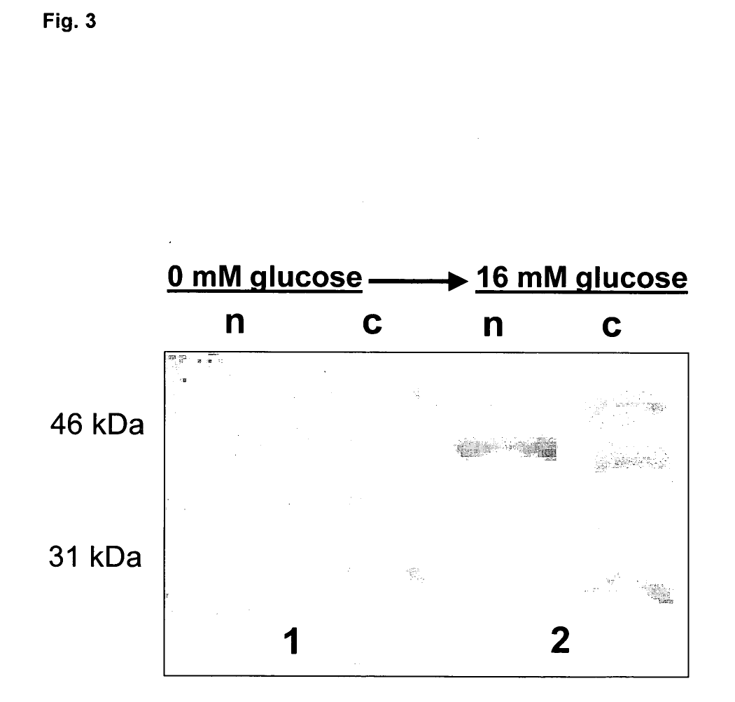

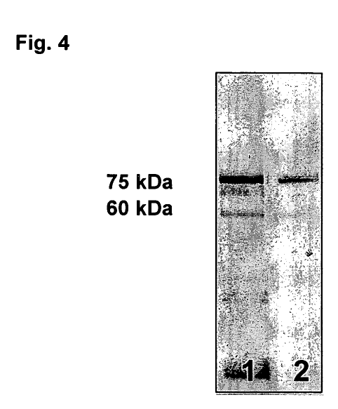

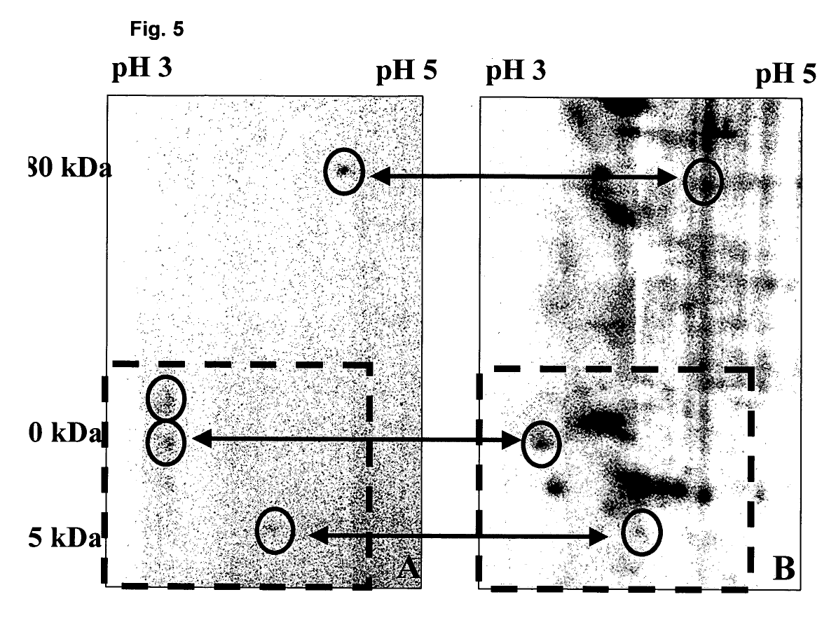

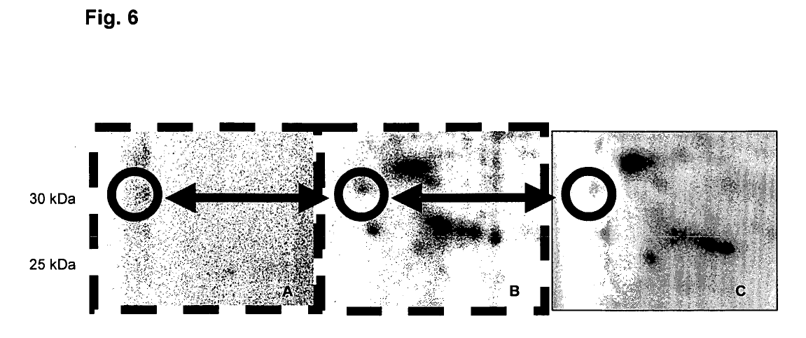

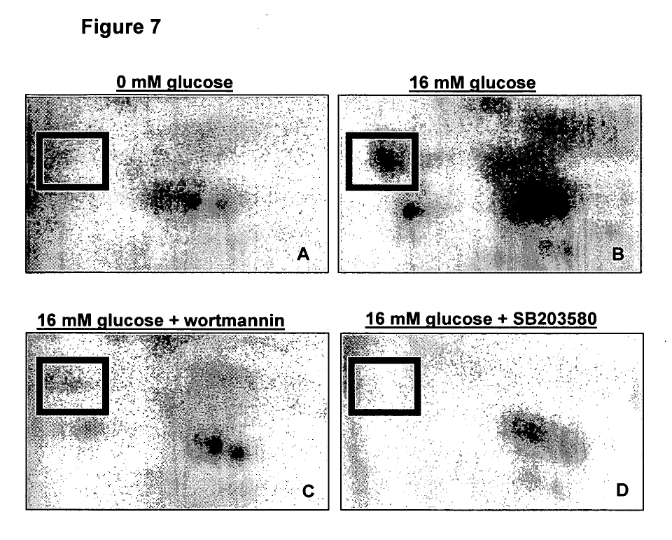

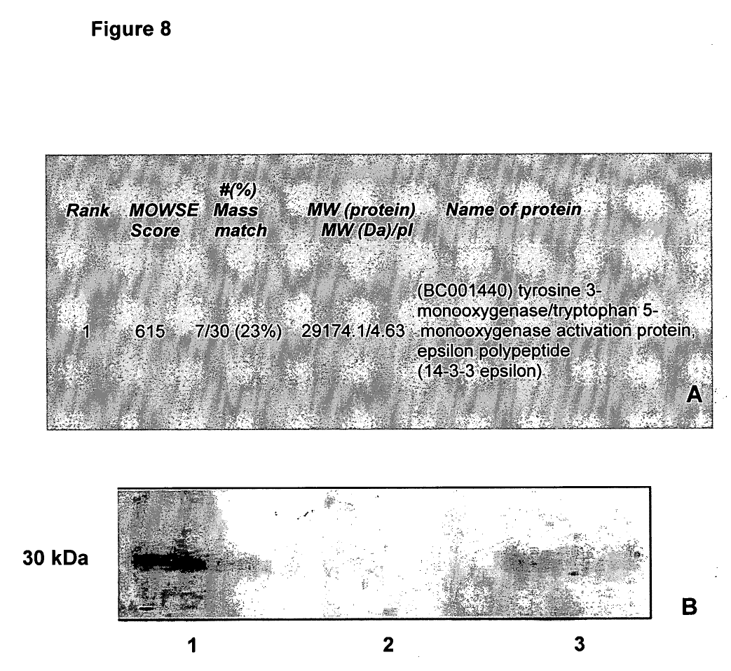

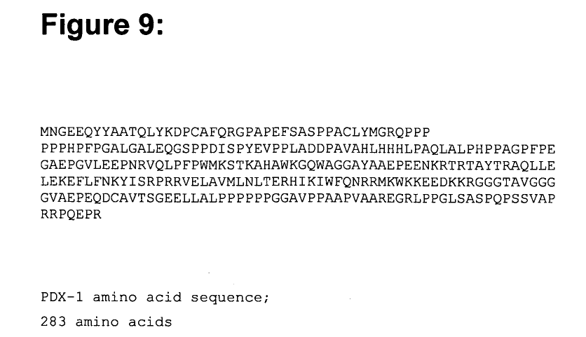

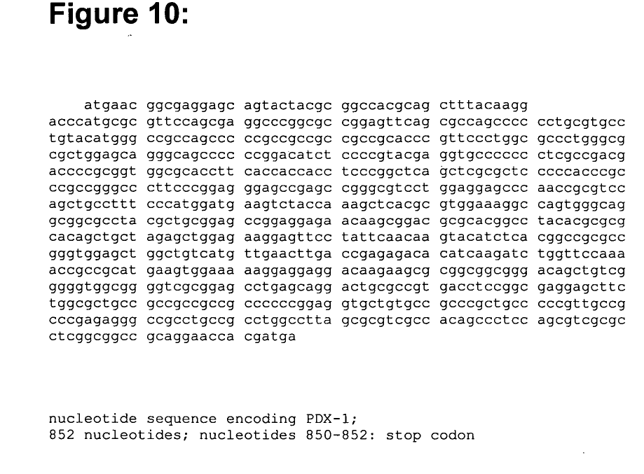

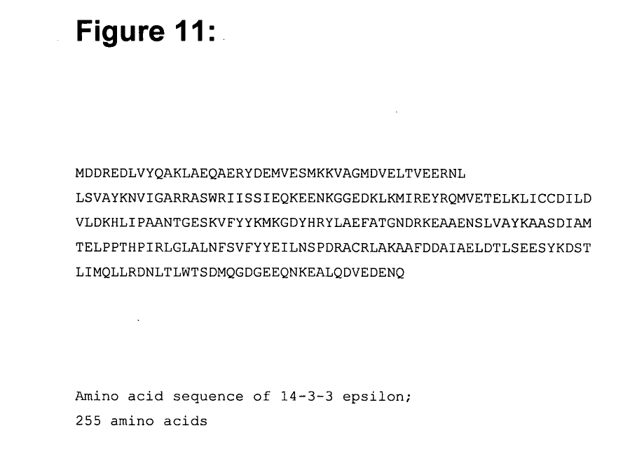

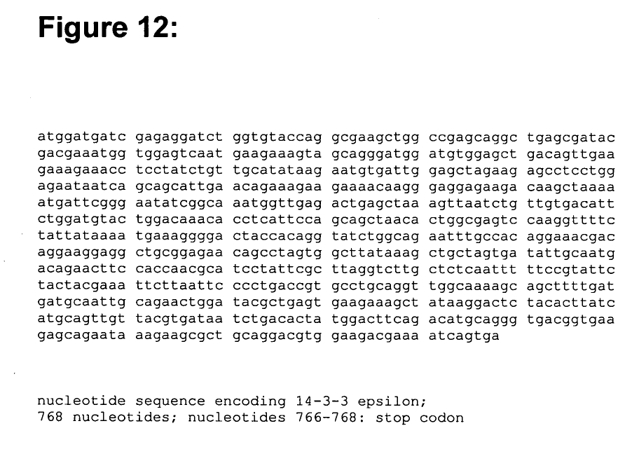

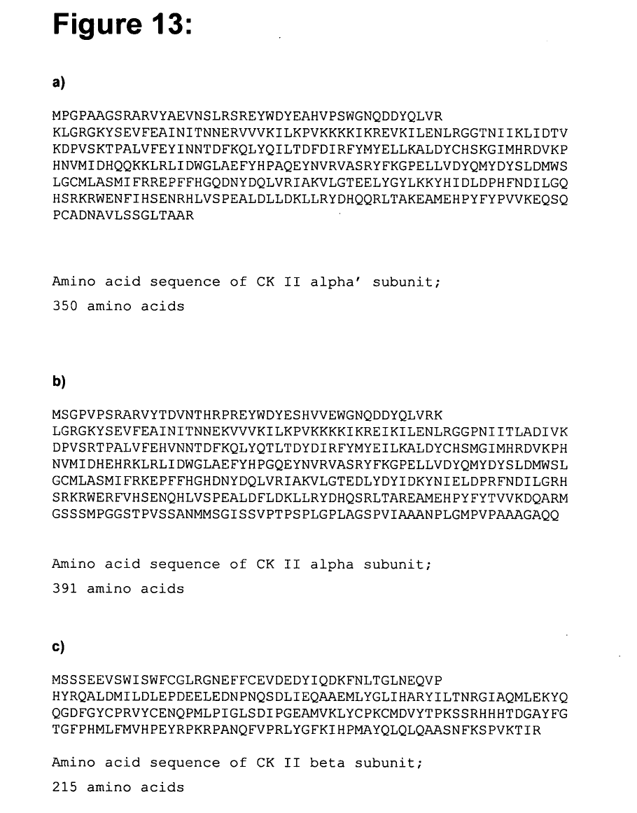

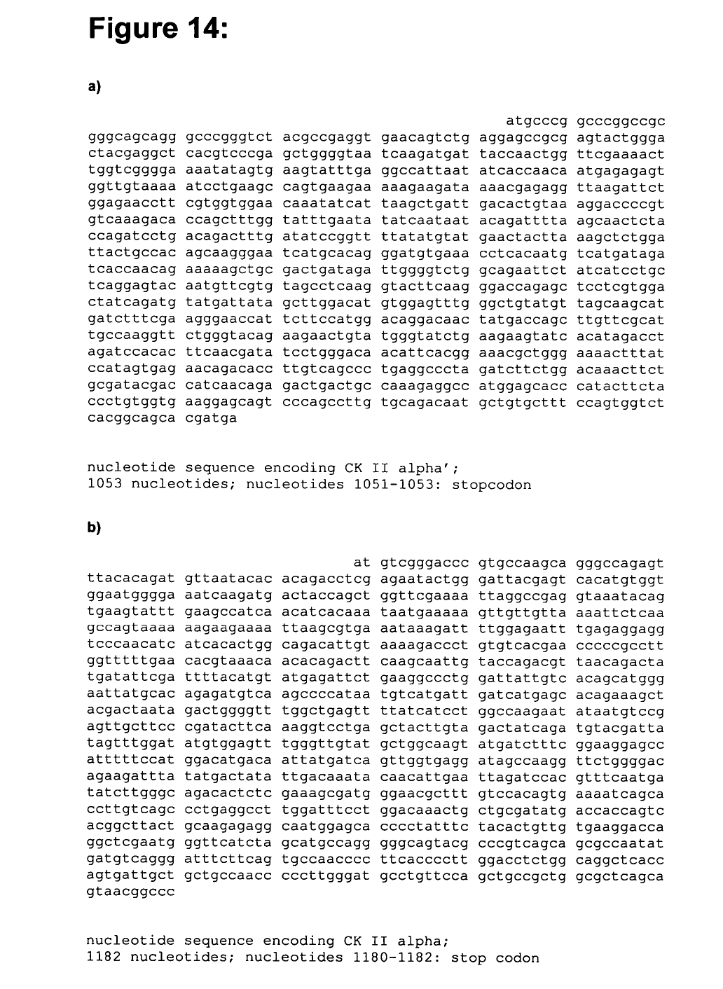

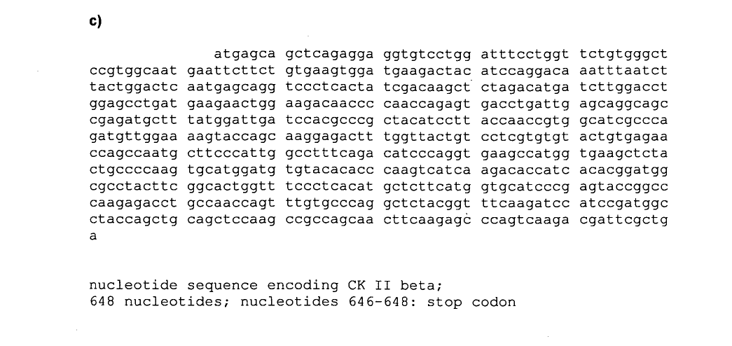

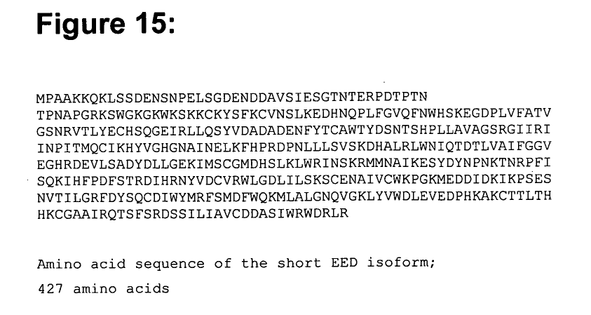

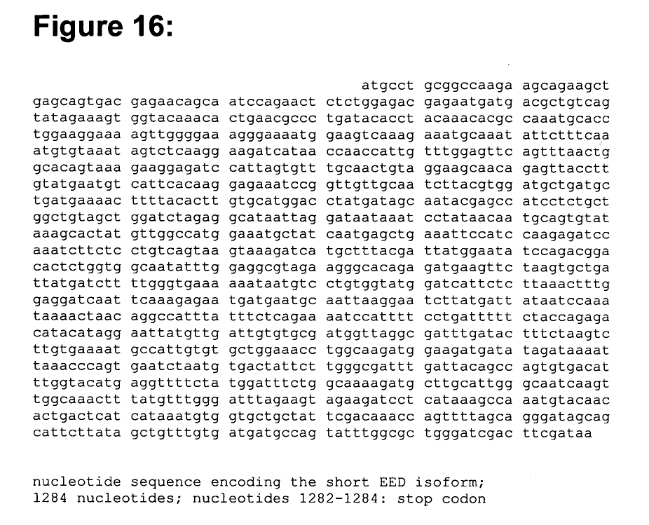

Bacterially expressed GST-PDX-1 reduces the amount of phosphoprotein in the supernatant of ³²P-labelled, glucose-stimulated MIN6 cells before two-dimensional gel electrophoresis.
A: The enlarged region of the 2D gel, which was subjected to autoradiography, shows the phosphoproteins obtained.
B: The enlarged region of a 2D gel, which was subjected to autoradiography, shows cytoplasmic phosphoproteins after acetone precipitation.
C: The enlarged region of a 2D gel, which was subjected to autoradiography, shows cytoplasmic phosphoproteins after acetone precipitation with subsequent incubation with GST-PDX-1 fusion proteins.

FIG. 7:

Changes in the phosphorylation state of the selected cytoplasmic protein as a reaction to glucose and various kinase inhibitors.

A-D:

Enlarged regions of 2D gels, which were subjected to autoradiography after successful acetone precipitation.
A: MIN6 cells were incubated for 8 hours at 0 mM glucose in Krebs Ringer Buffer.
B: The culture was transferred to 16 mM glucose.
C: The culture was transferred to 16 mM glucose in the presence of wortmannin (100 nM).
D: The culture was transferred to 16 mM glucose in the presence of SB203580 (10 μM).

FIG. 8:

Identification of 14-3-3-epsilon as an interaction partner of GST-PDX-1.
A: The results of the MS-Fit search yielded masses of a phosphoprotein that could be quantitatively precipitated from the cytoplasm of glucose-treated MIN6 cells using a bacterially expressed GST-PDX-1 protein.
B: Western blot using an anti-14-3-3 epsilon antiserum (Santa Cruz). Lane 1 (positive control): 30 μg cytoplasmic extract of glucose-treated MIN6 cells. Lane 2 (negative control): GST pulldown of 400 μg glucose-treated MIN6 cells. Lane 3: GST-PDX1 pulldown of 400 μg glucose-treated MIN6 cells.

FIG. 9:

Amino acid sequence of PDX1 (SEQ ID NO:2).

FIG. 10:

Nucleotide sequence encoding PDX-1 (SEQ ID NO:1).

FIG. 11:

Amino acid sequence of 14-3-3 epsilon (SEQ ID NO:10).

FIG. 12:

Nucleotide sequence encoding 14-3-3 epsilon (SEQ ID NO:9).

FIG. 13:

Amino acid sequence of the CK II subunits
a) Amino acid sequence of CK II alpha' (SEQ ID NO:4)
b) Amino acid sequence of CK II alpha (SEQ ID NO:6)
c) Amino acid sequence of CK II beta (SEQ ID NO:8)

FIG. 14:

Nucleotide sequence encoding the CK II subunits
a) Nucleotide sequence encoding CK II alpha' (SEQ ID NO:3)
b) Nucleotide sequence encoding CK II alpha (SEQ ID NO:5)
c) Nucleotide sequence encoding CK II beta (SEQ ID NO:7)

FIG. 15:

Nucleotide sequences of the short EED isoform (SEQ ID NO:11)

FIG. 16:

Amino acid sequences of the short EED isoform (SEQ ID NO:12)

1. Use of one or several proteins according to SEQ ID NO:4 and/or SEQ ID NO:6 and SEQ ID NO:8 (CK II), 10 (14-3-3 epsilon), EED or fragments of said proteins, for performing binding assays using a protein according to SEQ ID NO:2 (PDX-1), wherein the fragments bind to PDX1, for the identification of substances that influence (promote, inhibit, modulate) binding between the protein or proteins or fragment(s) and PDX-1.

2. A process for identifying substances that are suitable for influencing interaction of a protein according to SEQ ID NO:4 and/or SEQ ID NO:6 and SEQ ID NO:8 (CK II), 10 (14-3-3 epsilon), of the protein EED, or a fragment of said proteins, with the protein according to SEQ ID NO:2 (PDX-1), where
a) The protein according to SEQ ID NO:4 and/or SEQ ID NO:6 and SEQ ID NO:8 (CK II), 10 (14-3-3 epsilon), the protein EED, or a fragment of said proteins, is labelled,
b) The protein according to SEQ ID NO:2 (PDX-1) is labelled,
c) The labelled proteins from step a) and step b) are brought into contact with each other and a measurement is performed to determine the signal or signals of the label(s),
wherein the labels are so selected that interaction of labelled proteins from step a) and b) can be detected and distinguished from the isolated unlabelled proteins via alteration of the detection signal/detection signals,
d) The mixture from step c) is brought into contact with a substance to be examined, and
e) Another measurement is performed to determine the signal or signals of the label(s),
wherein the substance to be examined is a substance that influences the interaction, if the signal(s) of the label(s) measured in step e) differ(s) from the signal(s) of the label(s) measured in step c).

3. A process for identifying substances that are suitable for influencing interaction of a protein according to SEQ ID NO:4 and/or SEQ ID NO:6 and SEQ ID NO:8 (CK II), 10 (14-3-3 epsilon), EED, or a fragment of said proteins, with the protein according to SEQ ID NO:2 (PDX-1), where
a) The protein according to SEQ ID NO:4 and/or SEQ ID NO:6 and SEQ ID NO:8 (CK II), 10 (14-3-3 epsilon), the protein EED, or a fragment of said proteins, or
b) The protein according to SEQ ID NO:2 (PDX-1) is immobilised on a microtiter plate,
c) The other protein in question is labelled and brought into contact with the immobilised protein, wherein the presence of an interaction between the proteins mentioned in a) and b) is confirmed by detecting the labelling after performing corresponding washing steps,
d) The proteins are brought into contact with the substance to be examined,
wherein the substance to be examined is a substance influencing the interaction if, after addition of the substance to be examined and performing corresponding washing steps on the microtiter plates, the labelling is no longer detectable.

4. Use of a substance that influences the interaction of one or several proteins according to SEQ ID NO:4 and/or SEQ ID NO:6 and SEQ ID NO:8 (CK II), 10 (14-3-3 epsilon), EED or fragments of sai proteins, with the protein according to SEQ ID NO:2 (PDX-1), for manufacture of a pharmaceutical composition for the treatment of diseases that are characterised by decreased synthesis of insulin or that are accompanied by such decrease.

5. Use of a substance that
a) Modulates the activity of the protein according to SEQ ID NO:4 and/or SEQ ID NO:6 and SEQ ID NO:8 (CK II), 10 (14-3-3 epsilon), and/or the protein EED,
b) Binds to the protein according to SEQ ID NO:4 and/or SEQ ID NO:6 and SEQ ID NO:8 (CK II), 10 (14-3-3 epsilon), the protein EED or to a fragment of said proteins,
c) Phosphorylates the protein according to SEQ ID NO:2 (PDX1), SEQ ID:4 and SEQ ID NO:6 and SEQ ID NO:8 (CK II), 10 (14-3-3 epsilon) or the protein EED, or
d) Increases the proportion of the protein according to SEQ ID NO:4 and/or SEQ ID NO:6 and SEQ ID NO:8 (CK II),
for the manufacture of a pharmaceutical compound for the treatment of diseases that are characterised by decreased synthesis of insulin or that are accompanied by such decrease.

6. The use according to claims 4 and 5, wherein the disease that is characterised by decreased synthesis of insulin or that is accompanied by decreased synthesis of insulin is diabetes.

7. A process for the manufacture of a pharmaceutical composition for the treatment of diseases that are characterised by decreased synthesis of insulin or that are accompanied by such decrease, wherein a process according to claims 2 or 3 is performed, and the substance, which is identified as a substance influencing the interaction of a protein according to SEQ ID NO:4 and/or SEQ ID NO:6 and SEQ ID NO:8 (CK II), 10 (14-3-3 epsilon), EED or a fragment of said proteins with the protein according to SEQ ID NO:2 (PDX-1), is formulated into a pharmaceutical composition, using suitable excipients and/or carriers.

8. A pharmaceutical composition, wherein said compound contains a substance obtainable by a process according to claims 2 or 3 and pharmaceutically compatible excipients and/or carriers.

9. The use of one or several proteins according to SEQ ID NO:4 and/or SEQ ID NO:6 and SEQ ID NO:8 (CK II), 10 (14-3-3 epsilon), and EED, and/or fragments of said proteins, for the manufacture of a pharmaceutical preparation for the treatment of a disease that is characterised by decreased synthesis of insulin or that is accompanied by decreased synthesis of insulin is diabetes.

10. The use according to claim 9, wherein the disease is diabetes.

11. Use of one or several nucleic acids according to SEQ ID NO: 3 and/or 5 and 7 or 9, and/or one or several nucleic acids that encode EED, for the manufacture of a pharmaceutical preparation for modulating the synthesis of insulin in an individual.


SEQUENCE LISTING (12 sequences)

SEQ ID NO: 1 - DNA, 852 bp, Homo sapiens
  Stop codon (850)..(852): Nucleotide sequence of PDX-1
atgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatga

SEQ ID NO: 2 - protein, 283 aa, Homo sapiens
  Amino acid sequence of PDX-1
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 3 - DNA, 1182 bp, Homo sapiens
  Stop codon (1080)..(1082): Nucleotide sequence of CK II alpha subunit
atgtcgggacccgtgccaagcagggccagagtttacacagatgttaatacacacagacctcgagaatactgggattacgagtcacatgtggtggaatggggaaatcaagatgactaccagctggttcgaaaattaggccgaggtaaatacagtgaagtatttgaagccatcaacatcacaaataatgaaaaagttgttgttaaaattctcaagccagtaaaaaagaagaaaattaagcgtgaaataaagattttggagaatttgagaggaggtcccaacatcatcacactggcagacattgtaaaagaccctgtgtcacgaacccccgccttggtttttgaacacgtaaacaacacagacttcaagcaattgtaccagacgttaacagactatgatattcgattttacatgtatgagattctgaaggccctggattattgtcacagcatgggaattatgcacagagatgtcaagccccataatgtcatgattgatcatgagcacagaaagctacgactaatagactggggtttggctgagttttatcatcctggccaagaatataatgtccgagttgcttcccgatacttcaaaggtcctgagctacttgtagactatcagatgtacgattatagtttggatatgtggagtttgggttgtatgctggcaagtatgatctttcggaaggagccatttttccatggacatgacaattatgatcagttggtgaggatagccaaggttctggggacagaagatttatatgactatattgacaaatacaacattgaattagatccacgtttcaatgatatcttgggcagacactctcgaaagcgatgggaacgctttgtccacagtgaaaatcagcaccttgtcagccctgaggccttggatttcctggacaaactgctgcgatatgaccaccagtcacggcttactgcaagagaggcaatggagcacccctatttctacactgttgtgaaggaccaggctcgaatgggttcatctagcatgccagggggcagtacgcccgtcagcagcgccaatatgatgtcagggatttcttcagtgccaaccccttcaccccttggacctctggcaggctcaccagtgattgctgctgccaacccccttgggatgcctgttccagctgccgctggcgctcagcagtaacggccc

SEQ ID NO: 4 - protein, 391 aa, Homo sapiens
  Amino acid sequence of CKII alpha subunit
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 5 - DNA, 1053 bp, Homo sapiens
  Stop codon (1051)..(1053): Nucleotide sequence of CKII alpha? subunit
atgcccggcccggccgcgggcagcagggcccgggtctacgccgaggtgaacagtctgaggagccgcgagtactgggactacgaggctcacgtcccgagctggggtaatcaagatgattaccaactggttcgaaaacttggtcggggaaaatatagtgaagtatttgaggccattaatatcaccaacaatgagagagtggttgtaaaaatcctgaagccagtgaagaaaaagaagataaaacgagaggttaagattctggagaaccttcgtggtggaacaaatatcattaagctgattgacactgtaaaggaccccgtgtcaaagacaccagctttggtatttgaatatatcaataatacagattttaagcaactctaccagatcctgacagactttgatatccggttttatatgtatgaactacttaaagctctggattactgccacagcaagggaatcatgcacagggatgtgaaacctcacaatgtcatgatagatcaccaacagaaaaagctgcgactgatagattggggtctggcagaattctatcatcctgctcaggagtacaatgttcgtgtagcctcaaggtacttcaagggaccagagctcctcgtggactatcagatgtatgattatagcttggacatgtggagtttgggctgtatgttagcaagcatgatctttcgaagggaaccattcttccatggacaggacaactatgaccagcttgttcgcattgccaaggttctgggtacagaagaactgtatgggtatctgaagaagtatcacatagacctagatccacacttcaacgatatcctgggacaacattcacggaaacgctgggaaaactttatccatagtgagaacagacaccttgtcagccctgaggccctagatcttctggacaaacttctgcgatacgaccatcaacagagactgactgccaaagaggccatggagcacccatacttctaccctgtggtgaaggagcagtcccagccttgtgcagacaatgctgtgctttccagtggtctcacggcagcacgatga

SEQ ID NO: 6 - protein, 350 aa, Homo sapiens
  Amino acid sequence of CK II alpha? subunit
MPGPAAGSRARVYAEVNSLRSREYWDYEAHVPSWGNQDDYQLVRKLGRGKYSEVFEAINITNNERVVVKILKPVKKKKIKREVKILENLRGGTNIIKLIDTVKDPVSKTPALVFEYINNTDFKQLYQILTDFDIRFYMYELLKALDYCHSKGIMHRDVKPHNVMIDHQQKKLRLIDWGLAEFYHPAQEYNVRVASRYFKGPELLVDYQMYDYSLDMWSLGCMLASMIFRREPFFHGQDNYDQLVRIAKVLGTEELYGYLKKYHIDLDPHFNDILGQHSRKRWENFIHSENRHLVSPEALDLLDKLLRYDHQQRLTAKEAMEHPYFYPVVKEQSQPCADNAVLSSGLTAAR

SEQ ID NO: 7 - DNA, 648 bp, Homo sapiens
  Stop codon (646)..(648): Nucleotide sequence of CK II beta subunit
atgagcagctcagaggaggtgtcctggatttcctggttctgtgggctccgtggcaatgaattcttctgtgaagtggatgaagactacatccaggacaaatttaatcttactggactcaatgagcaggtccctcactatcgacaagctctagacatgatcttggacctggagcctgatgaagaactggaagacaaccccaaccagagtgacctgattgagcaggcagccgagatgctttatggattgatccacgcccgctacatccttaccaaccgtggcatcgcccagatgttggaaaagtaccagcaaggagactttggttactgtcctcgtgtgtactgtgagaaccagccaatgcttcccattggcctttcagacatcccaggtgaagccatggtgaagctctactgccccaagtgcatggatgtgtacacacccaagtcatcaagacaccatcacacggatggcgcctacttcggcactggtttccctcacatgctcttcatggtgcatcccgagtaccggcccaagagacctgccaaccagtttgtgcccaggctctacggtttcaagatccatccgatggcctaccagctgcagctccaagccgccagcaacttcaagagcccagtcaagacgattcgctga

SEQ ID NO: 8 - protein, 215 aa, Homo sapiens
  Amino acid sequence of CK II beta subunit
MSSSEEVSWISWFCGLRGNEFFCEVDEDYIQDKFNLTGLNEQVPHYRQALDMILDLEPDEELEDNPNQSDLIEQAAEMLYGLIHARYILTNRGIAQMLEKYQQGDFGYCPRVYCENQPMLPIGLSDIPGEAMVKLYCPKCMDVYTPKSSRHHHTDGAYFGTGFPHMLFMVHPEYRPKRPANQFVPRLYGFKIHPMAYQLQLQAASNFKSPVKTIR

SEQ ID NO: 9 - DNA, 768 bp, Homo sapiens
  Stop codon (766)..(768): Nucleotide sequence of 14-3-3 epsilon
atggatgatcgagaggatctggtgtaccaggcgaagctggccgagcaggctgagcgatacgacgaaatggtggagtcaatgaagaaagtagcagggatggatgtggagctgacagttgaagaaagaaacctcctatctgttgcatataagaatgtgattggagctagaagagcctcctggagaataatcagcagcattgaacagaaagaagaaaacaagggaggagaagacaagctaaaaatgattcgggaatatcggcaaatggttgagactgagctaaagttaatctgttgtgacattctggatgtactggacaaacacctcattccagcagctaacactggcgagtccaaggttttctattataaaatgaaaggggactaccacaggtatctggcagaatttgccacaggaaacgacaggaaggaggctgcggagaacagcctagtggcttataaagctgctagtgatattgcaatgacagaacttccaccaacgcatcctattcgcttaggtcttgctctcaatttttccgtattctactacgaaattcttaattcccctgaccgtgcctgcaggttggcaaaagcagcttttgatgatgcaattgcagaactggatacgctgagtgaagaaagctataaggactctacacttatcatgcagttgttacgtgataatctgacactatggacttcagacatgcagggtgacggtgaagagcagaataaagaagcgctgcaggacgtggaagacgaaaatcagtga

SEQ ID NO: 10 - protein, 255 aa, Homo sapiens
  Amino acid sequence of 14-3-3 epsilon
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 11 - DNA, 1284 bp, Homo sapiens
  Stop codon (1282)..(1284): Nucleotide sequence of the short isoform of EED
atgcctgcggccaagaagcagaagctgagcagtgacgagaacagcaatccagaactctctggagacgagaatgatgacgctgtcagtatagaaagtggtacaaacactgaacgccctgatacacctacaaacacgccaaatgcacctggaaggaaaagttggggaaagggaaaatggaagtcaaagaaatgcaaatattctttcaaatgtgtaaatagtctcaaggaagatcataaccaaccattgtttggagttcagtttaactggcacagtaaagaaggagatccattagtgtttgcaactgtaggaagcaacagagttaccttgtatgaatgtcattcacaaggagaaatccggttgttgcaatcttacgtggatgctgatgctgatgaaaacttttacacttgtgcatggacctatgatagcaatacgagccatcctctgctggctgtagctggatctagaggcataattaggataataaatcctataacaatgcagtgtataaagcactatgttggccatggaaatgctatcaatgagctgaaattccatccaagagatccaaatcttctcctgtcagtaagtaaagatcatgctttacgattatggaatatccagacggacactctggtggcaatatttggaggcgtagaagggcacagagatgaagttctaagtgctgattatgatcttttgggtgaaaaaataatgtcctgtggtatggatcattctcttaaactttggaggatcaattcaaagagaatgatgaatgcaattaaggaatcttatgattataatccaaataaaactaacaggccatttatttctcagaaaatccattttcctgatttttctaccagagacatacataggaattatgttgattgtgtgcgatggttaggcgatttgatactttctaagtcttgtgaaaatgccattgtgtgctggaaacctggcaagatggaagatgatatagataaaattaaacccagtgaatctaatgtgactattcttgggcgatttgattacagccagtgtgacatttggtacatgaggttttctatggatttctggcaaaagatgcttgcattgggcaatcaagttggcaaactttatgtttgggatttagaagtagaagatcctcataaagccaaatgtacaacactgactcatcataaatgtggtgctgctattcgacaaaccagttttagcagggatagcagcattcttatagctgtttgtgatgatgccagtatttggcgctgggatcgacttcgataa

SEQ ID NO: 12 - protein, 427 aa, Homo sapiens
  Amino acid sequence of the short isoform of EED
MPAAKKQKLSSDENSNPELSGDENDDAVSIESGTNTERPDTPTNTPNAPGRKSWGKGKWKSKKCKYSFKCVNSLKEDHNQPLFGVQFNWHSKEGDPLVFATVGSNRVTLYECHSQGEIRLLQSYVDADADENFYTCAWTYDSNTSHPLLAVAGSRGIIRIINPITMQCIKHYVGHGNAINELKFHPRDPNLLLSVSKDHALRLWNIQTDTLVAIFGGVEGHRDEVLSADYDLLGEKIMSCGMDHSLKLWRINSKRMMNAIKESYDYNPNKTNRPFISQKIHFPDFSTRDIHRNYVDCVRWLGDLILSKSCENAIVCWKPGKMEDDIDKIKPSESNVTILGRFDYSQCDIWYMRFSMDFWQKMLALGNQVGKLYVWDLEVEDPHKAKCTTLTHHKCGAAIRQTSFSRDSSILIAVCDDASIWRWDRLR